(19) United States
(12) Patent Application Publication
(10) Pub. No.: US 2025/0288358 A1
(43) Pub. Date: Sep. 18, 2025

(54) SYSTEMS FOR PROJECTION MAPPING AND MARKERLESS REGISTRATION FOR SURGICAL NAVIGATION, AND METHODS OF USE THEREOF

(71) Applicant: Illuminant Surgical, Inc., Palo Alto, CA (US)

(72) Inventors: Jhoneldrick S. MILLARES, Los Angeles, CA (US); James B. HU, Long Beach, CA (US); Philip A. O'CONNOR, Miami, FL (US); Brian M. NGUYEN, Culver City, CA (US); Ye Min AUNG, Amherst, MA (US); Thurein SOE, Brooklyn, NY (US); Joseph GU, Westford, MA (US); Enoch OMALE, Culver City, CA (US)

(73) Assignee: Illuminant Surgical, Inc., Palo Alto, CA (US)

(21) Appl. No.: 19/170,858

(22) Filed: Apr. 4, 2025

Related U.S. Application Data
(63) Continuation of application No. PCT/US2023/075968, filed on Oct. 4, 2023.
(60) Provisional application No. 63/413,121, filed on Oct. 4, 2022.

(51) Int. Cl. A61B 34/10; A61B 34/20; A61B 90/00

(52) U.S. Cl. CPC A61B 34/10; A61B 2034/105; A61B 2034/107; A61B 2034/2055; A61B 2090/364; A61B 2090/372; A61B 2090/374; A61B 2090/3762

(57) ABSTRACT

Some embodiments described herein relate to a method (e.g., a computer-implemented method) that includes receiving data associated with an operative field that includes a subject from an optical sensor. A three-dimensional (3D) virtual model associated with at least one of the subject or an object in the operative field can be accessed, and an observed mesh that includes a representation of the subject, based on the data received from the optical sensor can be defined. A virtual 3D environment, including the virtual model can be defined. The virtual model can be registered to the observed mesh, or the observed mesh can be registered to virtual model. A rendering of the virtual model can be projected, in real time, into the operative field such that the rendering of the virtual model is scaled and oriented relative to the at least one of the subject or the object in the real-world operative field as it appears in the virtual 3D environment.

### CROSS REFERENCE TO RELATED APPLICATIONS

This application is a continuation of International Patent Application No. PCT/US2023/075968, filed Oct. 4, 2023, which claims priority to and the benefit of U.S. provisional patent application No. 63/413,121, filed on Oct. 4, 2022, the entire contents of each of which are hereby incorporated by reference.

### BACKGROUND

Systems for surgical or non-surgical medical procedure guidance are used by healthcare professionals to help place and orient medical instruments with respect to patient anatomy. The usability of such systems is limited due to their use of 2D displays, such as computer LCD monitors, which require the physician to shift their view and attention away from the patient. There are some known systems that attempt to address the 2D display shortcoming by using projection mapping or augmented reality displays, but such systems are still limited by their reliance on fiducial markers placed on either the patient or the surgical instrument to enable tracking. Thus, there is a need in the surgical navigation field to create a novel and useful dynamic projection mapping system for surgical or non-surgical medical procedure guidance.

### SUMMARY

The present disclosure features dynamic projection mapping systems and methods of use thereof, for guidance in surgical and non-surgical medical procedures. In one aspect, the present disclosure provides methods of use of the system for markerless subject registration, medical instrument tracking, dynamic projection onto a subject surface, and/or the dynamic orthographic projection of sub-surface anatomy and/or geometry onto a subject surface. In some embodiments, the disclosed system and methods of use also comprise live and/or remote user collaboration. In some embodiments, a subject surface refers to a patient's body, e.g., patient's skin or other anatomical feature. In some embodiments, the subject surface is a surgical drape or other surface in the operative field. In further embodiments, the subject surface is a physical model, e.g., an anatomical model. The present system and methods of use may be used in surgical procedures as well as non-surgical medical procedures.

The dynamic projection mapping system of the present disclosure can include a “sense system,” a “compute system,” and/or a “display system.” The sense system can include one or more sensors. In some embodiments, the sensors can include one or more cameras or other optical detectors, such as RGB sensors, depth sensors (e.g., structured light sensors, time-of-flight sensors, stereo vision depth sensors), IR tracking cameras, and/or hyperspectral cameras. The compute system (e.g., computing hardware and software) can include input/output (I/O) device(s) that are used intraoperatively, such as a keyboard, mouse, foot pedals, or other intraoperative aids. The compute system can further include a computer processing system including software component(s) which are responsible for processing data in and out of the dynamic projection mapping system. In some embodiments, the display system of the present disclosure comprises one or more display monitors and one or more projector units.

In some embodiments, the sensors are co-mounted with the one or more projectors such that when the one or more projectors are moved, the sensors also move accordingly. This assembly may be referred to as the optical head. In some embodiments, said optical head is configured to be situated above the subject, e.g., patient.

The present disclosure further provides that the compute system can be a nexus for some and/or all input processing and output generation for the dynamic projection mapping system. In some embodiments, the compute system includes software component(s), also referred to as “modules”, which process inputs and develop outputs for dynamic projection onto a subject surface, e.g., a patient's body. In some embodiments, said software modules include: calibration; sensing; meshing; rendering; interaction; extrusion; registration; tracking; logging; and networking. In some embodiments, the compute system processes sensor input from the sense system, preoperative and/or intraoperative medical data, e.g., medical images or scans, and/or creates outputs for the display system, including visualizations, guidance, and other relevant data and user annotations. In some embodiments, the compute system enables users to annotate a digital representation of the operative field, wherein said annotations are added to the projected image/data. In some embodiments, the compute system further includes networking and communications with additional connected devices, e.g., the computers of remote users, intraoperative imaging devices, and/or secure databases. Said secure database may include patient medical records, and/or algorithms for processing data remotely (separately from the compute system) such as deep learning algorithms for segmentation or physical simulations to compute deformation of patient anatomy. In some embodiments, said networking and communication further comprises processing inputs from local and/or remote users or other connected computers/devices (e.g., tablet) and adding such data to the display system output.

The present disclosure further provides that the display system can receive an output from the compute system and display said output via the one or more display monitors and via the one or more projector units directly onto the subject surface. In some embodiments, the display output includes medical information, medical images, surgical guidance, and/or other medical and telemetric data. In some embodiments, surgical guidance includes (but is not necessarily limited to) displaying tracked instrument positions and/or orientations with respect to medical images, trajectory planning/labeling, verifying the position and orientation of extracted sub-surface geometry (from patient data), and numerical indicators such as a depth gauge for a tracked medical instrument approaching a target position. In some embodiments, the one or more display monitors are primarily used for presenting a graphical user interface (GUI) to the user for system setup and configuration. In some embodiments, the one or more projector units are primarily used for dynamic projection of the visual output directly onto a subject surface in the operative field, e.g., the patient's body (e.g., the patient's skin or other anatomical feature).

Some embodiments described herein relate to a method (e.g., a computer-implemented method) that includes receiving data associated with an operative field that includes a subject from an optical sensor. A three-dimensional (3D) virtual model associated with at least one of the subject or an object in the operative field can be accessed, and an observed mesh that includes a representation of the subject, based on the data received from the optical sensor can be defined. A virtual 3D environment, including the virtual model can be defined. The virtual model can be registered to the observed mesh, or the observed mesh can be registered to virtual model. A rendering of the virtual model can be projected, in real time, into the operative field such that the rendering of the virtual model is scaled and oriented relative to the at least one of the subject or the object in the real-world operative field as it appears in the virtual 3D environment.

Some embodiments described herein relate to a method (e.g., a computer-implemented method) that includes receiving data associated with an operative field that includes a subject and a surgical tool from an optical sensor. A three-dimensional (3D) virtual model associated with the surgical tool can be accessed. An observed mesh that includes a representation of the subject and a representation of the surgical tool can be defined based on the data received from the optical sensor. The observed mesh can be registered to a virtual 3D environment that includes the 3D virtual model associated with the surgical tool and a 3D virtual representation of the subject, or the virtual 3D environment can be registered to the observed mesh. A virtual camera can be defined in the virtual 3D environment, such that the virtual camera has a position and an orientation associated with a position and an orientation of the 3D virtual model of the surgical tool. A rendering of a virtual object can be projected in real time such that the rendering of the virtual object is scaled and oriented based on the position and the orientation of the surgical tool.

Some embodiments described herein relate to an apparatus that includes a housing, an optical sensor disposed within the housing, and a projector disposed within the housing. A processor can be operatively coupled to the optical sensor and the projector and configured to receive data from the optical sensor that associated with an operative field. The processor can define a virtual three-dimensional (3D) environment including a virtual representation of the subject and an annotation. The processor can register data received from the optical sensor to the virtual 3D environment or the virtual 3D environment to the data received from the optical sensor. The projector can receive, from the processor a signal to cause the projector to project a rendering of at least a portion of the virtual 3D environment that includes the annotation onto a surface of the subject.

### BRIEF DESCRIPTION OF THE DRAWINGS

FIG. 1 is schematic system diagram of a dynamic projection mapping system, according to an embodiment.

FIG. 2 is schematic diagram of a compute system, according to an embodiment.

FIG. 3 is a schematic diagram of a compute system, according to an embodiment.

FIG. 4 is a schematic diagram of a sense system, according to an embodiment.

FIG. 5 is a schematic diagram of a display system, according to an embodiment.

FIG. 6A is a cut-away view of an optical head assembly, according to an embodiment.

FIG. 6B is an isometric view of the optical head assembly of FIG. 6A.

FIG. 7 is a perspective view of a wheel-in assembly, according to an embodiment.

FIG. 8 shows an example of an optical head assembly in use, the resulting projected image, and the related 2D display monitor images.

FIG. 9 shows exemplary surgical indications for dynamic projection mapping.

FIG. 10 shows an example of the dynamic projection mapping system in use projecting medical information and visual annotations onto a model surface.

FIG. 11 shows an example of the dynamic projection mapping system in use projecting medical information onto a patient's body.

FIG. 12 is an illustration of an surgical tool tracking implementation, according to an embodiment.

FIG. 13 is an illustrated flow diagram of a method of generating a dynamic orthographic projection, according to an embodiment.

FIG. 14A depicts a flow chart of a method of capturing subject data and aligning medical image and sub-surface geometry, according to an embodiment.

FIG. 14B depicts a flow chart of a method that includes projecting annotations onto a surface of a subject, according to an embodiment.

### DETAILED DESCRIPTION

The present disclosure generally relates to a dynamic projection mapping system that can include: a sense system, a compute system, and/or a display system. The dynamic projection mapping system can, in some embodiments, be configured for: markerless subject registration, instrument tracking, real-time dynamic projection mapping of medical images and data, which can be continuously updated, and/or real-time local and remote user collaboration. In some embodiments, the sense system can be used to capture live information about the operative field, such as data pertaining to position and orientation of the subject and medical instrument(s). In some embodiments, said data also includes 2D images of the subject and operative field. In some embodiments, a compute system can process the data from the sense system and convert said data into outputs for the display system. In some embodiments, said outputs can include annotations such as: 2D and/or 3D visualizations, guidance, trajectories, annotations drawn with a separate input device (such as a touch display), and/or other relevant data, one or more of which can be projected onto the subject surface. In some embodiments, the compute system can also be used to process annotations from local and remote users and add these to the output for the display system. In some embodiments, the display system can be used to present the 2D and 3D visualizations and other data to the user. In some embodiments, these visualizations are continuously updated throughout the procedure.

### Hardware

Embodiments described herein can include a dynamic projection mapping system for surgical or non-surgical procedure guidance. The dynamic projection mapping system can include a sense system, a compute system, and/or a display system that work together in concert for dynamic projection mapping of medical information and other surgical data. It should be understood that the sense system, compute system, and display system (and/or components thereof) may not be physically and/or logically distinct. Similarly stated, the sense system, compute system, and display system are generally described as different systems for ease of description, but may be partially and/or completely integrated and/or logically and/or physically subdivided into additional systems.

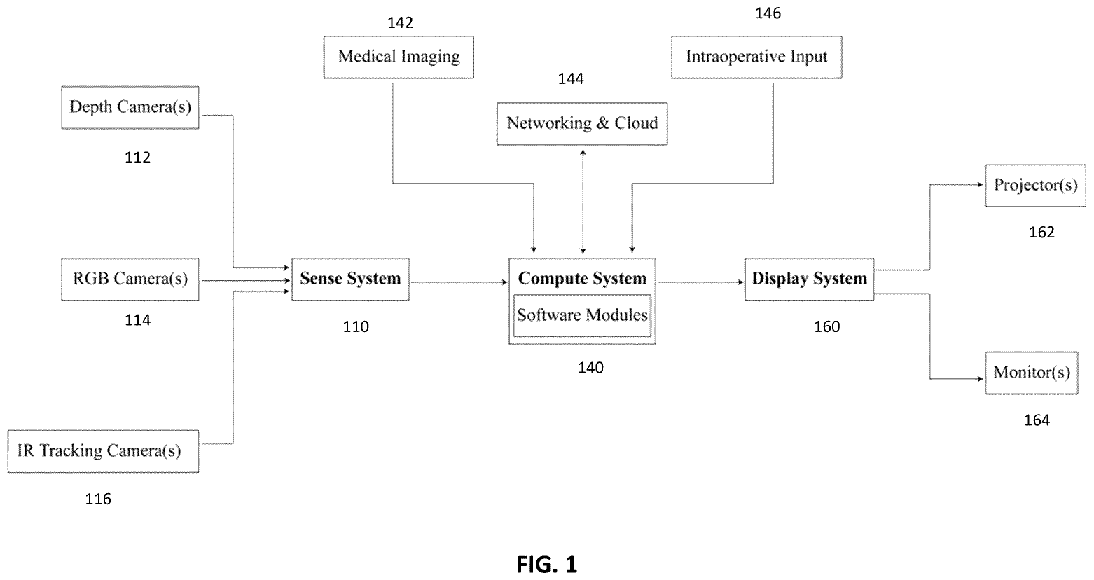

FIG. 1. is a schematic system diagram of a dynamic projection mapping system, according to an embodiment. The system can include a sense system 110, a compute system 140, and/or a display system 160. In some embodiments, the sense system 110 can provide data to the compute system 140, and the compute system 140 can provide an output developed from data received from the sense system 110 to the display system 160. In some embodiments, this output can be projected directly onto a subject surface in the operative field. In some embodiments, said subject surface is one or more of the following: a patient's body, e.g., a patient's skin and/or other anatomical feature; a physical model, e.g., an educational model; or a surgical drape or other surface present in the operative field.

As shown in FIGS. 1 and 4, the sense system 110 can include one or more sensors. In particular, the sense system 110 typically includes one or more optical detectors (also referred to herein as cameras). For example, the sense system 110 can include one or more a depth camera(s) 112, RGB camera(s) 114, and/or infrared tracking camera(s) 116. Examples of depth cameras 112 include structured light sensors, time-of-flight sensors, light detection and ranging (LIDAR) emitters and/or detectors, and/or stereo vision depth sensors. The sense system 110 can also include hyperspectral sensors and/or cameras (e.g., thermal IR sensors, UV sensors, etc.). It should be understood that while FIGS. 1 and 4 generally depict discrete sensors for ease of illustration and discussion, in some embodiments a sensor can perform more than one function, such as an RGBD (RGB and Depth) sensor.

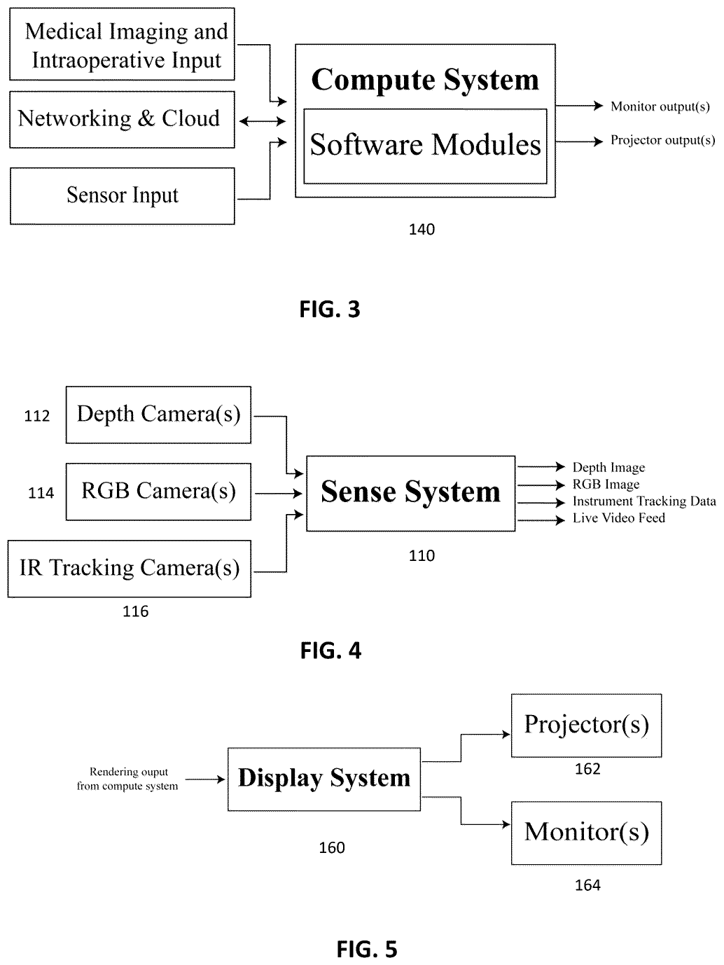

Compute system 140 can be operable to receive and process input data received from the sense system 110 and define, create, and/or maintain outputs sent to the display system 160 (or other system(s)). The compute system 140 of the present disclosure comprises the computing hardware and software components. Similarly stated, the compute system can include one or more processors and a memories. The memory can be non-transitory and can store code configured to be executed by the processor to cause the processor to perform computational functions described herein. In some embodiments, sense system inputs are received from the sense system 110, and developed outputs are sent to the display system 160. In some embodiments, the compute system 140 can include a server-class computer, a desktop computer, a laptop computer, a tablet computer, and/or any other suitable compute system and/or related equipment communicatively coupled to the sense system and/or the display system (e.g., via a network and/or the internet). The compute system 140 can be co-located with and/or remote from the sense system and/or the display system. In some embodiments, the compute system can be and/or use distributed computing resources (e.g., the cloud). In some embodiments, the compute system 140 can be integrated with the sense system 110 and/or the display system 160 (e.g., the compute system and the sense system and/or display system may be physically contained in the same housing). In some embodiments, the compute system 140 can include input/output device(s) associated with the operative field, which can be used intraoperatively (e.g., keyboard, mouse, foot pedal, or other intraoperative aids). In some embodiments, the compute system 110 can include input/output device(s) used preoperatively (e.g., to plan a procedure) and/or remotely (e.g., to receive guidance from an individual not in the operating theater). An example of a schematic illustration of the compute system 140 can be seen in FIG. 3.

The display system 160 can be configured to display medical information, surgical guidance, and/or other medical and/or telemetric data to the user (e.g., a surgeon or other medical professional). In some embodiments, the display system 160 receives an output from the compute system 140, to cause the data and/or visualizations to be displayed. The display system will typically include one or more projectors 162. In some embodiments, the display system can include one or more display monitors 164, as can be seen in FIG. 5. In some embodiments, 2D data is projected dynamically from a projector 162 onto the subject surface such that the projected data conforms to the shape and contours of the subject surface. In some embodiments, the one or more display monitors are configured for presenting a graphical user interface (GUI) for system setup and configuration. In some embodiments, the one or more projectors 162 are configured for dynamic projection of a visual output directly onto a subject surface in the operative field. In some embodiments, the one or more display monitors 164 are mounted in the operative field. In some embodiments, a 2D display is provided to remote users comprising a live intraoperative view of the operative field.

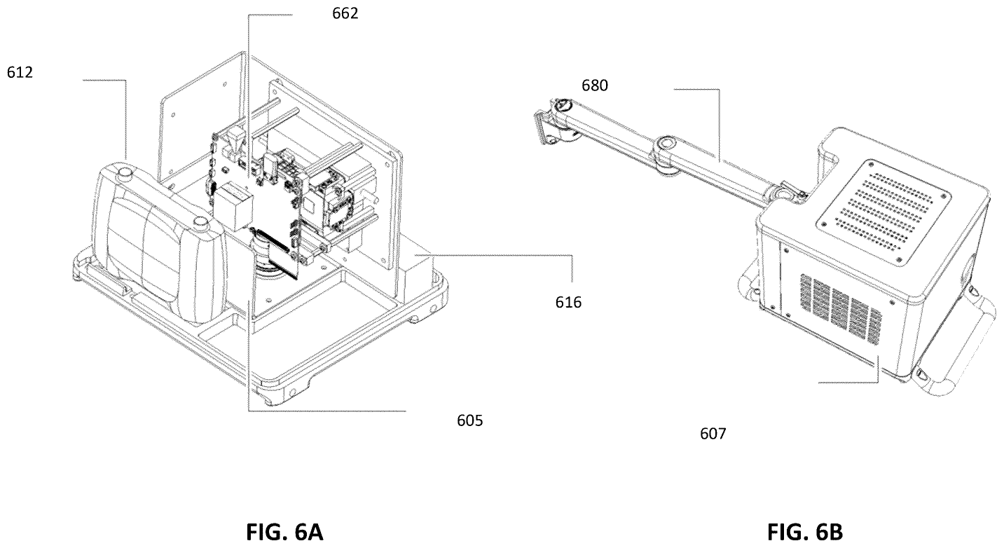

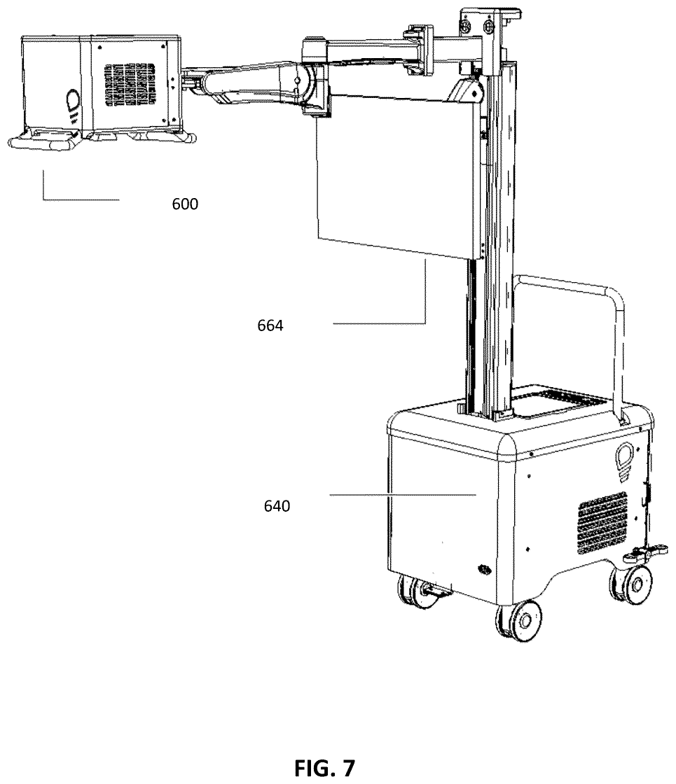

In some embodiments, the sensor(s) comprising the sense system 110 are co-mounted and/or encased with the projector unit(s) such that when the one or more projectors moves, the associated sensor(s) also move accordingly. An embodiment of an optical head 600, containing sensor(s) 610 and projector(s) 660, is shown in FIGS. 6A and 6B. FIG. 6A depicts the optical head 600 with a portion of the housing removed to reveal internal components. A depth camera 612 and projector 662 can each be mounted to a frame 605. As shown in FIG. 6A, optics (e.g., lenses, detectors, etc.) are configured to detect and/or project downward out of a bottom of the housing 607. The optical head 600 may be disposed above the subject, e.g., patient. In some embodiments, as shown in FIG. 6B, the optical head is mounted on an articulating arm 680 to allow freedom of movement, e.g., positioning, tilting, and swiveling about multiple axes. In some embodiments, the optical head is mounted on a wheelable cart, in conjunction with the display monitor(s) and computer. Such a wheelable cart can be set up, for example, as follows: the display monitor(s) 664 are attached above the base of the cart, along with a platform for keyboard and mouse; the optical head 600 is attached to the base of the cart via an articulating arm 680, as described above; and an optional backup battery is stored below the base. An example of this configuration can be seen in FIG. 7. In such a cart-based system, the compute system 640 can be disposed in a base of the cart. In some embodiments, the optical head 600 is mounted permanently in the operative field, such as via a wall-or ceiling-mounted articulating arm.

### Software

The compute system can act a hub for input processing and output development associated with the dynamic projection mapping system. In some embodiments, the compute system processes input from the sense system and develops outputs to the display system, wherein said outputs include visualizations, surgical guidance and other relevant medical and/or telemetric data. In some embodiments, the compute system input processing includes processing data from pre-operative and/or intraoperative medical imaging, e.g., CT scans, MRIs, x-rays, etc. Processing such data can include pixel/voxel segmentation and labeling of anatomical structures within the medical images, volumetric or surface reconstruction of anatomical structures from such segmentations resulting in a 3D model, and post-processing of the 3D model such as filtering noise and closing holes in the 3D model. In some embodiments, the compute system further comprises networking and communicating with other associated computers, via a local network or secure internet connection. In some embodiments, these other associated computers may be used to connect remote users who provide pre-operative annotations, real-time annotations or guidance, and/or data centers/cloud computing solutions which can provide further processing capability, data or models. In some embodiments, this includes: algorithms for processing sensor data; anatomical models; and/or medical data for assisting a user intraoperatively or for practice purposes.

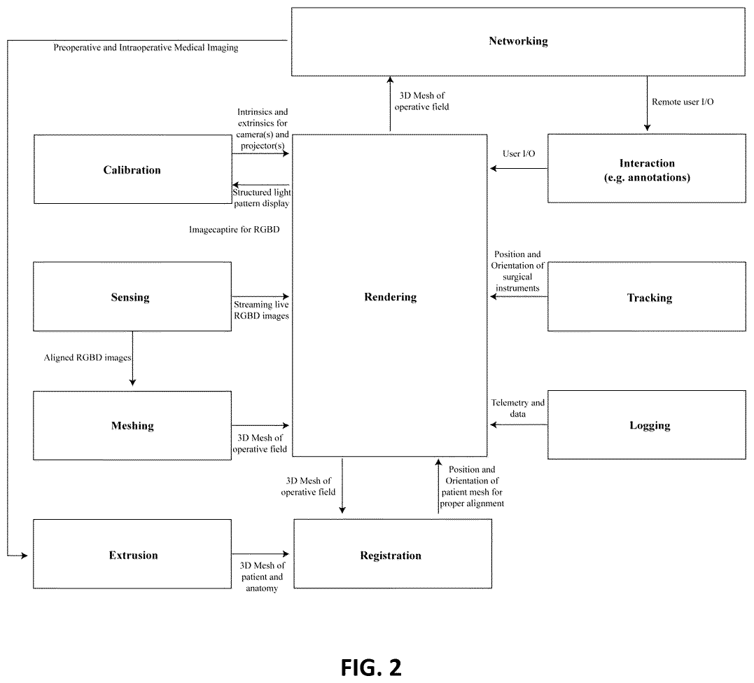

Embodiments described herein can include software-implemented methods and/or techniques. Code stored in non-transitory processor-readable memory can include instructions configured to cause on or more processors to carry out various tasks or operations. For ease of understanding and description, it can be useful to discuss software-related modules that enable discrete functions of the dynamic projection mapping system, as can be seen in FIG. 2. Is should be understood, however, that in practice, “modules” described herein may not be physically and/or logically distinct. Similarly stated, tasks, operations and/or functions discussed in the context of a certain software module may be partially and/or completely integrated with other operations, tasks, functions, and/or “modules,” and/or logically and/or physically subdivided into additional modules. As shown in FIG. 2, the dynamic projection mapping system can include the following software modules: a calibration module; a sensing module; a meshing module; a rendering module; an interaction module; an extrusion module; a registration module; a tracking module; a logging module; and a networking module. It should be understood that the various software modules are shown and described separately for ease of discussion, and other implementations where various functions of such modules are differently distributed are possible.

In some embodiments, the calibration module is configured to perform geometric calibration of optical systems, such as sensors, cameras, projectors, and/or display monitors. In some embodiments, geometric calibration includes identification of the relative positioning of optical systems in the operative field (e.g., sensors, cameras, projectors, display monitors), determination, calculation, and/or estimation of optical systems' intrinsic and/or extrinsic properties, and/or distortion removal from optical systems. This can allow for accurate translation of geometric data between sensor and/or virtual and real-world coordinate systems with a high fidelity. In some embodiments, calibration is performed either at the time of assembly, or at the time of installation. In some embodiments, calibration is performed after the sensors are moved relative to the projector(s). In some embodiments, the calibration procedure is carried out between sensors by capturing multiple poses of a known pattern, such as a chessboard with known dimensions, within the view of sensors. Using a standard pinhole camera model, one can solve a system of linear equations using the positions of visual features on the chessboard to find the intrinsic and extrinsic parameters of sensors that detect the known pattern, yielding the relative position of such sensors in the operative field. In some embodiments, the calibration procedure is carried out between a sensor and a projector by capturing multiple poses of an object with known dimensions and features, such as a chessboard of black and white squares with squares of known dimensions, within view of the sensor and the projector. The projector projects a known pattern, or a series of patterns, onto the chessboard while the sensor captures each pattern. Using the principle of structured light, the positions of visual features on the chessboard can be found from the perspective of a projector. Using a standard pinhole camera model, one can solve a system of linear equations using the positions of visual features on the chessboard to find the intrinsic and extrinsic parameters of the projector, yielding the relative position of projectors in the operative field. In some embodiments, lens distortion parameters, such as those within the Brown-Conrady distortion model, can be solved for using chessboard calibration. Such distortion parameters can be used to remove distortions from optical systems.

In some embodiments, the sensing module is configured to obtain sensor data from sensors/cameras. In some embodiments, this sensor data is in the form of aligned RGBD images, wherein each color pixel has an associated depth. In some embodiments, a hyperspectral camera may be used wherein each color pixel is associated with additional channels corresponding to hyperspectral image values. In some embodiments, the sensing module uses the sensor manufacturer's public facing application programming interface (API) to communicate with a specific sensor. In some embodiments, the sensing module is a software abstraction which reveals a standard API to interact with an array of different sensors, potentially of different models or manufacturers, contained in the system in a standard manner. In other embodiments, the sensing module can obtain sensor data from sensors and/or cameras using any other suitable communications interface, such as closed, proprietary, and/or custom interfaces. In some instances, the sensing module can obtain raw sensor data. In some embodiments, preprocessing may occur in the sensing module.

In some embodiments, the meshing module is configured to reconstruct RGBD and/or point cloud data from the sensing module into a solid 3D mesh, referred to herein as an observed mesh. In some embodiments, the meshing module can perform the reconstruction in real time, enabling live updates of the observed mesh. A “mesh” is a collection of vertices and triangular faces (composed of 3 connected vertices), as well as any associated properties for each vertex or face (e.g., color or other data). In some embodiments, the meshing module utilizes a truncated signed distance function (TSDF) data structure to create the 3D mesh. In some embodiments, a different meshing algorithm is used. In some embodiments, the meshing module can, similarly to the sensing module, apply various transformations to captured meshes, such as smoothing, filtering, and hole-filling operations.

In some embodiments, the rendering module enables the display, transformation, and manipulation of visual data. More specifically, this module may create an intraoperative visualization window for a display monitor, as well as provide display data for the projector(s) of the display system. In some embodiments, the intraoperative display window shows real-time guidance data for 2D display. In some embodiments, this module further manages the display of data to remote users. In some embodiments, the rendering module is the central nexus of data for other modules in the system. For example, the rendering module may display the operative field meshes from the meshing module; utilize the projector parameters from the calibration module to synthesize display data for the projector(s); display live data from the sensing module; communicate data to and from associated computers via the networking module; utilize aligned patient mesh data from the registration module and instrument positioning data from the tracking module; respond to user input and output from the interaction module; and store relevant data via the logging module.

In some embodiments, the rendering module uses a 3D real-time rendering engine or software package to render mesh data in a virtual 3D environment. In some embodiments, the 3D real-time rendering engine manages the creation, destruction, and visibility of graphical windows which show different views and objects in the rendered virtual environment. In some embodiments, the rendering module synthesizes the images to be displayed by the projectors by creating a virtual scene which mimics the physical scene captured by the sensors in the sense system. This virtual scene is a digital twin of the physical, real-world scene-objects within this virtual scene are scaled and positioned such that their size and relative positions measured in the virtual world's 3D coordinate system correspond to their real-world size and relative positions. The size and relative position of such objects are typically captured by a 3D sensor, such as a structured light scanner or a time-of-flight sensor. This virtual scene can also contain color information about each object, which is usually sourced from an RGB sensor. In some embodiments, virtual objects with real-world counterparts such as pedicle screws are added to the scene as they're added to the real-world environment. The virtual scene is created to facilitate accurate alignment and high-fidelity display of projected images from the real-world projector onto the real-world scene. For example, by creating a virtual camera with equivalent intrinsic parameters (e.g., focal length, principal point, skew) and extrinsic parameters (e.g., position, orientation) as the projector in the real-world scene, the virtual images captured by this virtual camera can be used to create a rendering that will closely approximate the field of view of the real-world projector. This means any parts of virtual objects captured within the view of the virtual camera will correspond to real-world objects that are illuminated by the real-world projector. In fact, a pixel-to-pixel mapping can be created, whereby a pixel in the virtual camera will map to a pixel in the real-world projector. For example, the top left pixel in the virtual camera's image will be the top left pixel in the real-world projector's projected image.

Once the virtual environment is created, virtual objects (having real-world counterparts) can be annotated with additional information, such as anatomical information, deep structures in the human body, incision guidelines, entry point visualizations etc. Such annotations may not have real-world counterparts but may be added onto the surface geometry of virtual objects such that they conform to the virtual objects. The projector can then project a rendering of the virtual scene including the annotations onto the real-world scene. Because the annotations conform to the surface geometry of the virtual objects, when a rendering of the objects and/or annotations are projected out onto the corresponding physical objects themselves, the annotations will conform to the surface geometry of the real-world objects. This technique, sometimes known as projection mapping, enables digital manipulation of the visual properties of physical objects. In some embodiments the rendering module is responsible for rendering an image of sub-surface geometry, such as the spine or other internal anatomy, onto the subject surface. This projection is both dynamic and orthographic. It is dynamic, in that it is updated in real-time and is responsive to user input, and is orthographic in that the virtual camera viewing the geometry uses an orthographic projection model rather than a perspective projection model. This results in a spatially meaningful dynamic projection which is view independent.

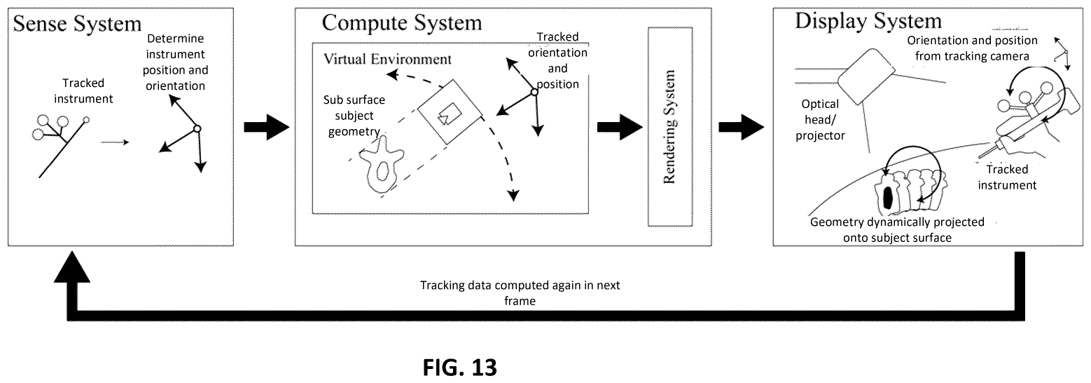

As seen in FIG. 13, in some embodiments, a dynamic, orthographic projection can be created based on the orientation of an instrument. A sense system can receive data associated with an instrument. For example, the instrument can include fiducial markers or otherwise be identifiable. Based on data received from the sense system, a position and orientation of the instrument can be calculated. A model of the instrument can be generated in a virtual 3D environment (e.g., a model can be accessed or retrieved from a library of instruments) that corresponds to the position and orientation of the physical surgical instrument. Similarly stated, a mesh (or model) in the 3D virtual environment can be aligned and registered with an observed mesh (e.g., as detected by the sense system) of the surgical instrument in the operative field (or vice versa. A virtual camera can be defined that is associated with the virtual model of the surgical instrument, such that the viewing direction of the virtual camera is given by the orientation of the medical instrument, currently being tracked by the sensing module. In this way, as the virtual model of the surgical instrument moves (in response to the sense system detecting movement of the physical surgical instrument), so too does the field of view of the virtual camera. Annotations can then be rendered in the virtual 3D environment from the perspective of the virtual camera. For example, a virtual projector that corresponds to or is collocated with the virtual camera can project annotations from the perspective of the virtual surgical tool. For example, sub-surface anatomy such as bone structure, vasculature, etc., which can be obtained from a model created based on pre-operative and/or intraoperative medical imaging, can be (e.g., orthographically) projected in the virtual 3D environment onto a virtual surface of the subject. A second virtual camera that corresponds to the projector, can then orthographically capture an image/render of the 3D virtual environment, including the virtual annotations. The field of view of this second camera can then be projected from the physical projector. In this way, the annotations (e.g., sub-surface anatomy) can be projected from the point of view of the tool rather than any one user. In this embodiment any geometry that appears beneath the tool tip on the subject surface is, in reality, directly in the line of sight of the tool, without any viewing distortion based on the distance to the surface (due to the orthogonal projection in the virtual 3D environment).

It may be desirable for virtual objects within the virtual environment to be transparent to certain virtual cameras, of which the rendering module may comprise many. In this way only objects to be displayed by the projector (e.g., annotations) are visible in the virtual world to the virtual camera, which prevents reprojection of the virtual object onto itself in the physical world. Similarly stated, in an instance in which a spine annotation is to be projected onto a real-world torso, in the virtual scene the virtual torso may be transparent such that the projector does not project an image of the (virtual) torso onto the (real-world) torso. Typically, this means making all virtual objects transparent in the virtual scene, and keeping only the annotations visible. Additionally, it may be desirable for the background of the virtual scene to be black, since projectors are additive displays.

In some embodiments, the interaction module processes local user input and output (I/O) to the system. Said interactions may take the form of keyboard commands, computer mouse actions, touch display input, and foot pedal devices in the operative field. In some embodiments, interaction further includes processing remote user I/O. In some embodiments, the interaction module utilizes the operating system's libraries to access local user I/O. In some embodiments, the interaction model utilizes internet protocols such as HTTPS or TCP/IP communication in order to receive and send remote user I/O.

In some embodiments, the extrusion module extracts 3D anatomical meshes (also referred to herein as subject reference mesh(es)) from medical imaging data of the subject/patient. In some embodiments, said 3D meshes are utilized for subject registration. In some embodiments, extrusion is manual or semi-automated, including segmenting image layers using user-defined annotations and thresholds to create a 3D mesh. In some embodiments, extrusion is fully automated, utilizing a machine learning module to process and perform segmentation, wherein the final mesh is reviewed and accuracy confirmed by the user. In other embodiments, input data is passed via the networking module to be extruded remotely by an algorithm running on the cloud. In some embodiments, the extrusion module receives DICOM (Digital Imaging and Communications in Medicine) images via the file system or network as input to the extrusion process. In some embodiments, extrusion is performed by annotating pixels or voxels within slices of imaging data, where an annotation includes a tag or color which signifies which anatomical feature such a pixel or voxel belongs to. For example, an abdominal CT scan may be annotated by clicking all pixels or voxels which correspond to bony tissue across each scan slice. In some embodiments, these annotated pixels or voxels are used as inputs into 3D surface reconstruction algorithms, such as the marching cubes algorithm, to create a 3D mesh.

In some embodiments, the registration module aligns the subject reference mesh from the extrusion module and an observed mesh of the subject in the operative field from the meshing module. Subject registration may be manual, semi-automated, or fully automated (e.g., markerless). In some embodiments, manual registration comprises the user directly changing the position and orientation of the reference mesh, via GUI controls or keyboard inputs, such that it aligns with the observed mesh. In other embodiments, semi-automated registration comprises utilizing markers on the subject to indicate proper position and orientation. In a preferred embodiment, fully automated markerless registration comprises utilizing point cloud registration techniques for surface alignment and registration between the reference mesh and the observed mesh. In some embodiments, the meshes or their corresponding point clouds are sampled to use as reference points in registration algorithms. In some embodiments, fully automated markerless registration begins by first computing geometric features of some or all surface points in the reference and observed mesh. For example, a Fast Point Feature Histogram (FPFH) feature can be calculated for all points in the observed mesh. In some embodiments, the automated registration algorithm finds correspondences between points and features across the two point clouds (subject reference mesh and observed mesh). For example, a correspondence between FPFH features can be found by finding the nearest neighbor in its multidimensional vector space. In some embodiments, the 3D transformation including a translation and a rotation can be found by applying random sample consensus (RANSAC) algorithms to the correspondences and fitting a translation and rotation to minimize the Euclidean distance between such points. Finding such a translation and rotation results in proper alignment of the reference and observed mesh. In some embodiments, an iterative closest point (ICP) algorithm can be applied to find or refine the resulting 3D transformation. Point cloud registration techniques may be assisted by information already known about the scene such as an initial estimate of the orientations of both the reference and observed meshes, known as priors. These priors can be static, user defined or inferred by the compute system via input from the sense system.

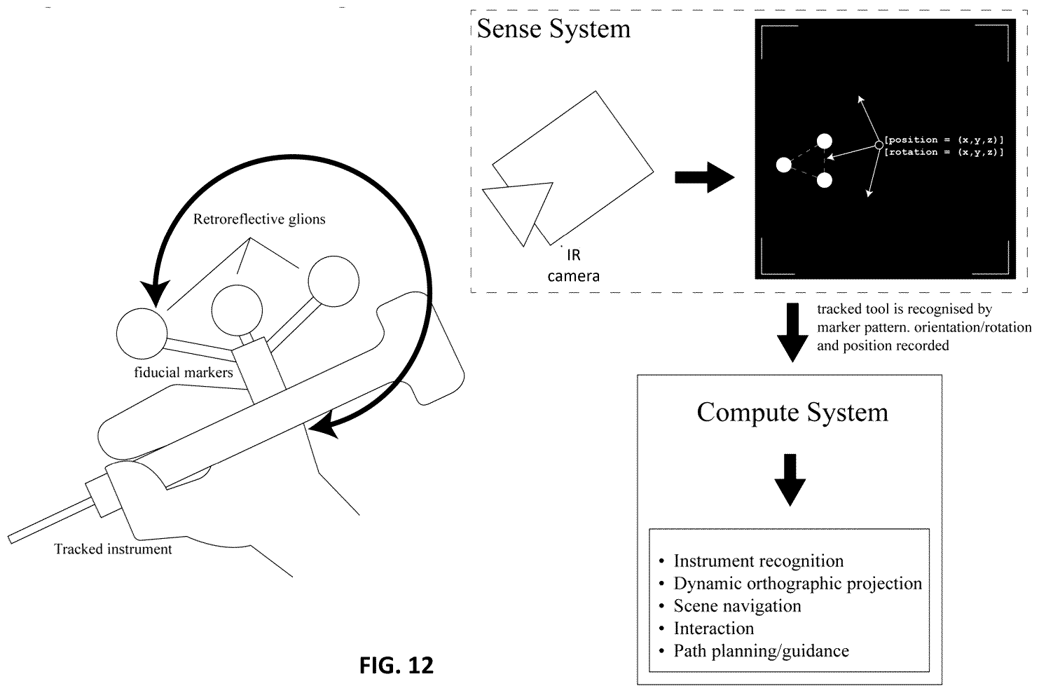

In some embodiments, the tracking module tracks medical instruments in 3D space utilizing 3D models of the instruments in use and template matching algorithms to localize instrument position and orientation in the operative field. Such template matching algorithms could automatically recognize instruments that have unique geometrical features with respect to one another. For example, such geometrical features can include protrusions, handles, attachment points, and other distinct geometry. In some embodiments, the tracking module uses a real-time optimized version of the methods used in the registration module to continuously update the registration of the patient and instruments, resulting in tracking. In some embodiments, the tracking module uses the 3D CAD model or a high resolution and high-fidelity 3D scan of the instrument as the reference mesh for registration. In other embodiments, the tracking module tracks medical instruments in 3D space utilizing active or passive fiducial markers such as retro-reflective spheres (or glions) tracked by an infrared optical tracking system as depicted in FIG. 12. In some embodiments, the output of the tracking module consists of one or more instruments' true world positions and orientation, with each instrument identified as a specific type of instrument. Recognition of instruments in such embodiments would require different spatial arrangements of infrared tracking markers affixed onto each instrument, where the system associates a specific spatial arrangement of such markers with a specific instrument type.

In some embodiments, the logging module can save telemetric, medical and/or other relevant data of a procedure. Said data may include medical images, intraoperative images, video, 3D depth data, and other relevant data. In some embodiments, logs can be simple informational, warning, and error text data emitted by the application. In some embodiments, data logs can be used to reproduce procedures, further offline analysis, or for a follow up procedure/demo where the user would like to resume progress.

In some embodiments, the networking module handles communications between computers connected with the system. In some embodiments, connected computers include the following: those of remote users; datacenter computers which provide relevant procedure data that is not subject-specific; computers which are providing subject records, e.g., medical records; and other associated computers. In some embodiments, the networking module uses standard internet protocols and system libraries thereof, such as TCP/IP and sockets, to transmit and receive data.

### Methods of Use

The present disclosure features methods of use of the disclosed system for dynamic projection mapping of medical information for surgical and non-surgical procedure guidance. In some embodiments, the disclosed system is used for dynamic projection mapping of medical information directly onto a subject surface, for use in an operative field. In some embodiments, said dynamic projection mapping further comprises markerless subject registration and tracking, instrument tracking, and real-time user collaboration and annotation. A user may be local in the operative field or they may be remotely viewing the procedure. In some embodiments, wherein the user is remote, they are provided with a digital representation of the operative field. In some embodiments, a plurality of software modules, as part of the compute system, enable the functionality of the system.

FIG. 1 depicts a schematic system architecture, comprising both the hardware and software components, according to an embodiment. In some embodiments, sensor(s) collect data about the operative field and provide such data to the sense system. In some embodiments, the sensor(s) include one or more cameras. In some embodiments, the sensor(s) comprises one or more of the following: RGB sensors or cameras; depth sensors or cameras (e.g., structured light sensors, time-of-flight sensors, and/or stereo vision depth sensors); IR tracking sensors or cameras; and hyperspectral sensors or cameras (e.g., thermal IR sensors, UV sensors, etc.). In some embodiments, the data collected includes continuously updated, live information about the operative field, such as position and orientation of the subject and medical instruments. In some embodiments, update rates from sensors used for tool tracking would typically be greater than or equal to 20 frames per second. In some embodiments, the accuracy of sensors used for tool tracking would include less than 1 mm of error. In some embodiments, update rates from sensors used to capture images of the surgical environment would typically be greater than or equal to 1 frame per second. In some embodiments, the accuracy of 3D sensors used for capturing images of the surgical environment would include less than 1 mm of error. In some embodiments, the sense system sends the collected data to the compute system, wherein the sensor input data is used to construct the virtual scene and/or virtual objects and/or develop outputs for the display system. In some embodiments, said outputs include one or more of the following: visualizations, guidance, and other medical or telemetric data. In some embodiments, these outputs include 2D representations for display on one or more display monitors and 3D representations for dynamic projection by the one or more projector units. Such projector units would typically have refresh rates greater than 20 frames per second, with resolutions typically at least 1920×1080 pixels, and with brightness greater than 3000 ANSI lumens.

FIG. 2 depicts a schematic illustration of software modules, according to an embodiment. In some embodiments the software modules are employed in the following manners: the calibration module calibrates optical systems; the sensing module configures sensors and provides an interface to obtain sensor data; the meshing module creates a solid 3D mesh from the sensing module data; the rendering module is used for the display, transformation and manipulation of all visual data; the extrusion module extracts 3D anatomical meshes from medical imaging data; the registration module aligns the patient reference mesh from the extrusion module and the live patient mesh from the meshing module; the interaction module handles all user input and output; the tracking module tracks the medical instruments in a 3D field; the networking module handles communications between computers on a secure network, e.g., remote user computers; and the logging module saves telemetry and other relevant data, e.g., images, video, 3D depth data, etc. In some embodiments, multiple modules are used at one time. In another aspect, some of the software modules may be employed multiple times over the course of a procedure, and not necessarily in the order described. In some embodiments, some of the software modules can be reused to reproduce results or recreate previously recorded procedures or demonstrations/sessions.

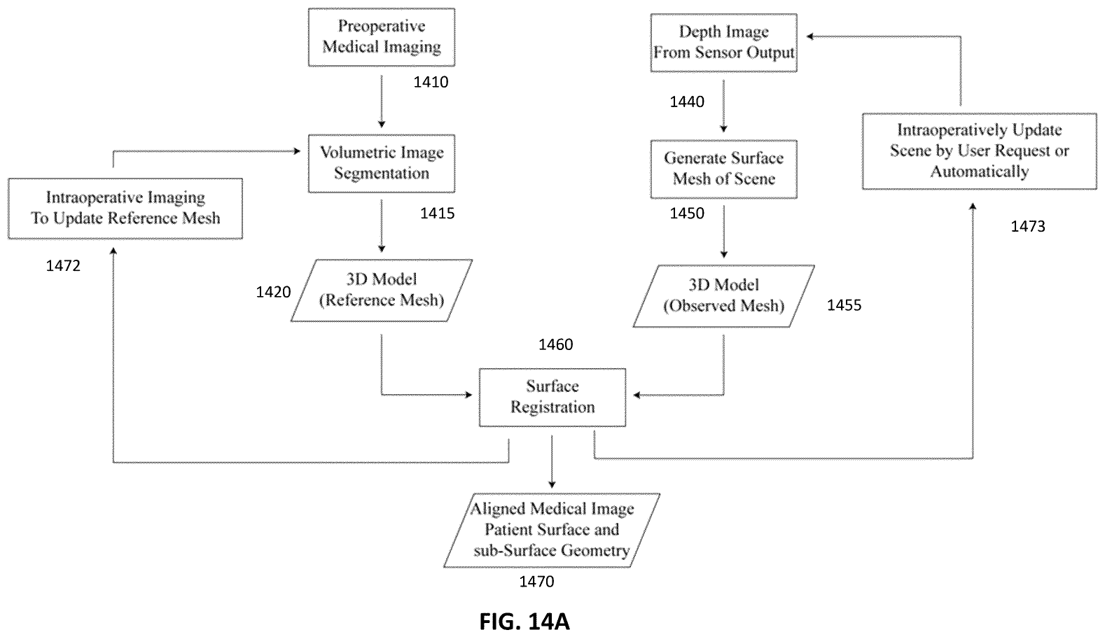

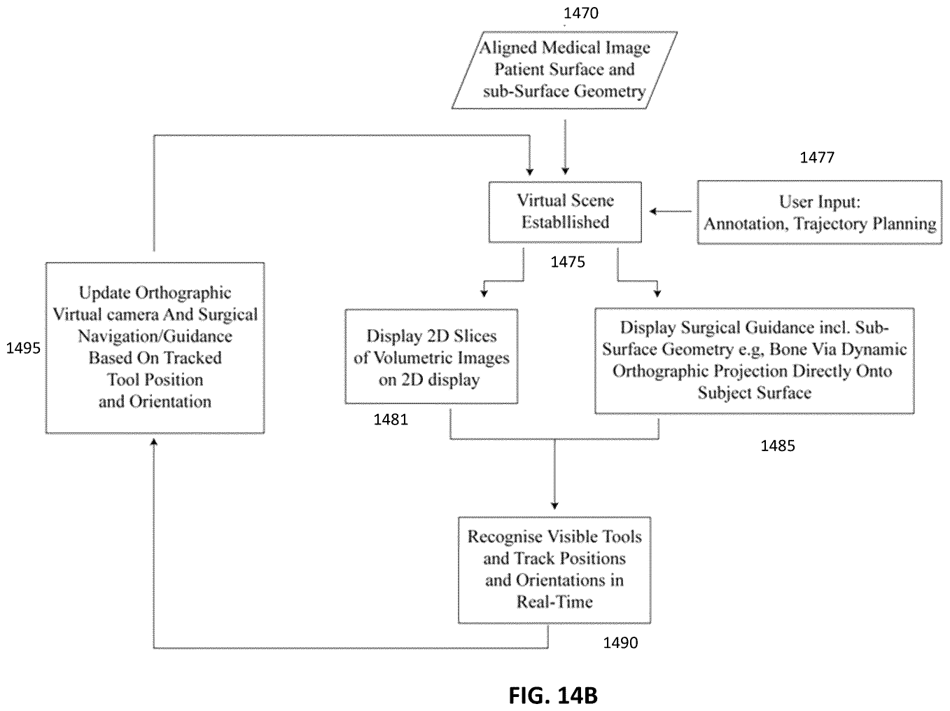

FIG. 14A depicts a flow chart of a method of capturing subject data and aligning medical image and sub-surface geometry, according to an embodiment. At 1410, data associated with medical imaging (e.g., CT scan, PET scan, ultrasound, x-ray, MRI, etc.) can be received. The data received at 1410 can, in some embodiments, include individual 2D “slices.” At 1415, image data can be volumetrically segments to generate a 3D model (or reference mesh) 1420 of at least a portion of the subject based on the medical imaging and volumetric segmentation. For example, the medical imaging data can be pixel-or voxel-wise segmented with anatomical labels, which are then used to generate a 3D anatomical model of the subject via a surface reconstruction process, such as the marching cubes algorithm. The 3D model can virtually represent at least a portion of a subject's anatomy including sub-surface anatomical features. In some embodiments, a data representing a 3D model of the subject can be received directly. Similarly stated some medical imaging devices and accompanying processing tasks can be operable to produce a reference model suitable for surface registration, at 1440 or a reference model can be retrieved from a library or database.

At 1440, data from an optical sensor, such as a 3D camera can be received. The optical sensor can be a component of a head unit and configured to image an operative field, which will typically include a subject. The data received at 1440 includes depth information. At 1450 a surface mesh of the field of view of the optical sensor can be generated and a 3D model 1455 (also referred to herein as an observed mesh) can be defined based on the data received from the optical sensor. In this way, the observed mesh can be a representation of the actual physical state of the operative field, surgical tools within the operative field, and/or the subject.

At 1460, the reference mesh defined at 1420 can be registered to the observed mesh defined at 1455, or vice versa, to create an aligned medical image patent surface and sub-surface geometry 1470. Registration can involve conforming the virtual model (the “reference mesh”) of the subject and/or other objects within the operative field and/or subject to the actual observed state of the subject/operative field. For example, in an automatic and markerless registration system point cloud registration techniques can be used to align, various observed surface anatomical features, such as shoulders, hips, back mid-point, or sub-surface features, such as exposed bone(s), to the reference mesh. The observed mesh can be adjusted or otherwise manipulated such that the position and/or orientation of corresponding virtual surface or sub-surface features conform to the actual physical observed positions and orientations. As another example, in some implementations, markers can be placed on the physical subject, surgical tools, or other salient points in the operative field. Such markers can be detected via the data from the optical sensor and corresponding predefined reference points of the reference mesh can be conformed to match the actual physical observed positions and orientations of the markers. In some embodiments, this registration process is manual wherein the user manually adjusts the position and orientation of the reference mesh such that it aligns with the observed subject.

The aligned medical image patient surface and sub-surface geometry 1470 can thus represent a virtual model that corresponds to and has position(s) and orientation(s) that match the physical subject and/or other objects in the operating field. In some embodiments, intraoperative imaging can be performed, and data from such intraoperative imaging can be used, at 1472, to update the reference mesh. In this way, the position and/or orientation of the subject and/or other objects within the operative field can be updated when they move, shift position and/or orientation relative to the optical sensor, and or are modified or removed via surgical intervention. Data from the intraoperative imaging can be volumetrically segmented, at 1415, and the reference mesh can be updated, at 1420 in a manner similar to the preoperative medical image data. In other embodiments, preoperative imaging data may not be available and/or intraoperative imaging can be solely used to generate the reference mesh.

FIG. 14B depicts a flow chart of a method that includes projecting annotations onto a surface of a subject, according to an embodiment. The method of FIG. 14B can be a continuation of the method of FIG. 14A. Specifically, the method of FIG. 14B can begin with aligned medical image patient surface and sub-surface geometry 1470 that can represent a virtual model that corresponds to and has position(s) and orientation(s) that match the physical subject and/or other objects in the operating field. A virtual scene, or virtual 3D environment containing the virtual model can be established and/or defined at 1475. The virtual 3D environment can defined in any suitable 3D rendering engine.

In some embodiments, at 1481 a live 2D image of the virtual scene/environment can be provided to one or more display monitors, in order to pass through live intraoperative video feed(s) for viewing and annotation by local or remote users. Said 2D display may further include a visualization of the operative field and real-time guidance data. In some embodiments, the 2D display is displayed on one or more monitors locally. In some embodiments, the 2D display is transmitted to remote computers.

In some embodiments, user input, at 1477, can allow local and/or remote users to annotate the 2D display, for example, using a keyboard, stylus, or other suitable input device. Such annotations are transmitted to the compute system and incorporated into the virtual 3D environment. In some embodiments, users are able to directly annotate the 3D visualization. In some embodiments, such annotations can be made by displaying the 3D model on a monitor and using a touchscreen or a mouse to click and drag on surfaces on the 3D model and digitally painting onto the 3D model. In some embodiments, such annotations can take the form of straight lines, curved lines, geometric shapes, text boxes, and image boxes which can be positioned and dragged about the surface of the 3D model via said touchscreen or mouse interfaces. In some embodiments, such annotations are rendered as either 2D or 3D objects alongside the 3D model within a 3D engine. In some embodiments, annotations are added and updated throughout the procedure. In some embodiments, viewing and annotating of the display comprises local and/or remote users being able to “paint” lines directly onto the 2D or 3D visualization, and said annotations then being displayed within the 3D projection.

At 1485, a rendering of the virtual scene can be projected from the optical head into the operative field. As discussed above, in some embodiments, projecting the rendering of the virtual field, at 1485 can include defining a virtual camera in the virtual environment having intrinsic and/or extrinsic properties corresponding to a physical projector. Thus, a virtual field of view taken from the virtual camera can correspond to the projector's field of view, such that the rendering projected into the operative field is scaled and oriented relative to the subject and/or object in the real-world environment, as it appears in the virtual environment.

In some embodiments, at 1490, surgical tools, medical instruments, and/or other objects can be recognized and/or tracked using the optical sensor. As discussed above, the real-world position and/or orientation of such objects can mapped to the virtual environment and the compute system can then update a corresponding position and orientation of a counterpart virtual object in the virtual environment. In some embodiments, fiducial markers can be attached to such objects, which can facilitate tracking via the sense system with processing of inputs occurring in the software's sensing module. In some embodiments, medical instruments have no such sensor and said instrument tracking comprises: processing sensor data to monitor the position, orientation and trajectory of said instruments; and utilizing 3D models of said instruments and template matching algorithms to localize instrument position, orientation, and trajectory in the operative field. Tracking instruments allow the system to display the instrument's position, orientation, and trajectory with respect to patient anatomy to the medical staff to guide staff members in the placement and movement of such instruments. In some embodiments, such observed position, orientation, and trajectory information can be compared to a desired position, orientation, and trajectory. Differences between the observed and desired position, orientation, and trajectories can be displayed (directly projected on the subject surface and/or output to one or more connected 2D displays) to medical staff to allow them to correct their manipulation of the instruments.

In some embodiments, intraoperative aids are also in communication with the disclosed system. Such intraoperative aids may include keyboard commands, mouse actions, foot pedal devices, or other similar such aid. These aids allow medical staff to interact with the hardware or software system, such as selecting the active medical instrument to track and model, or enabling system features.

In some embodiments, a virtual camera (different from the virtual camera associated with the physical projector) can be associated with a surgical tool or other object within the virtual environment. In such embodiments, annotations, surgical navigation, and/or guidance can be updated based on a position and/or orientation of the physical surgical tool based on the corresponding position and/or orientation of the virtual surgical tool and associated virtual camera, at 1495. As discussed above, annotations can be virtually orthographically projected onto the virtual model of the subject in the virtual environment from the point of view of the virtual camera associated with the virtual tool. Then, at 1485, a rendering of the virtual environment, including any annotations projected from the perspective of the tool-associated virtual camera, can be captured using the projector-associated virtual camera and that updated rendering can be emitted from the physical projector.

### Exemplary Uses

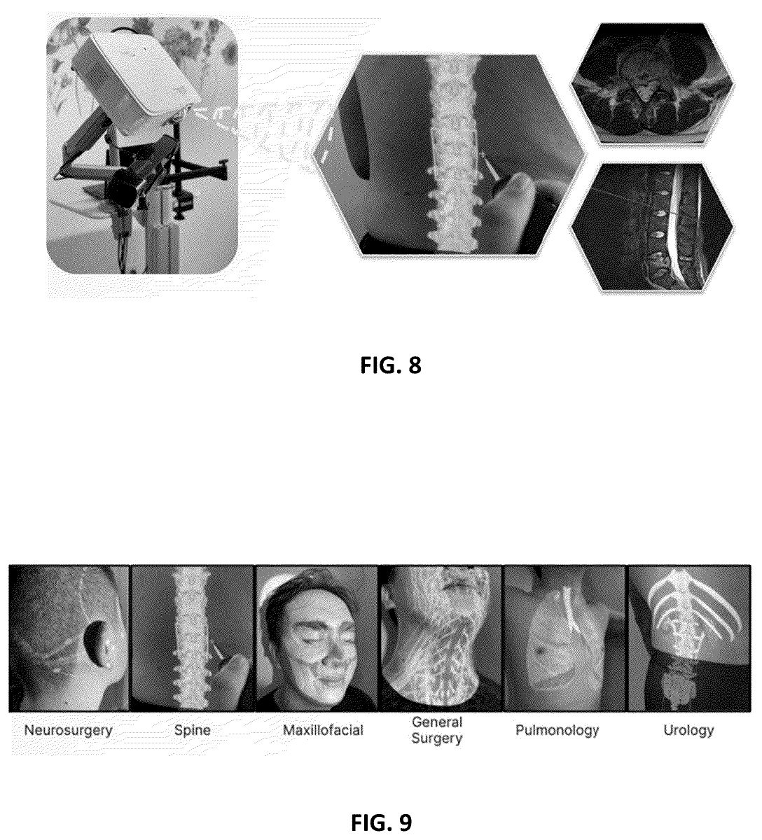

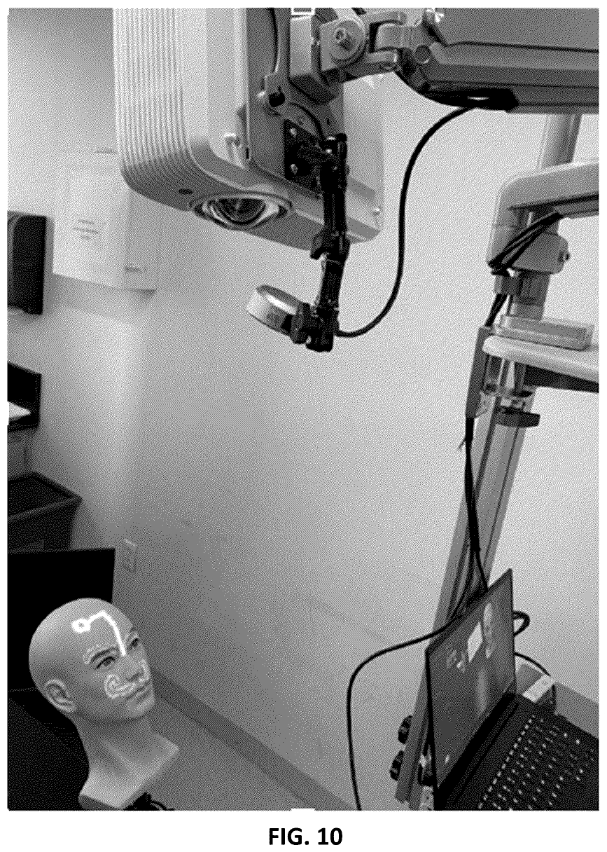

The present disclosure provides a non-exhaustive list of exemplary use indications. FIGS. 8-11 depict projected displays on a subject surface, e.g., patient anatomy, in various exemplary fields of use. The disclosed system and methods of use may be used for remote proctoring and annotation as a method of training users. In some embodiments, the disclosed system and methods of use allow a remote proctor to add guidance to projected surgical navigation and/or guidance displays, wherein the local users are practicing on an anatomical model or other practice subject surface. In some embodiments, remote users are able to navigate the 3D environment on a 2D display and annotate their display such that the annotations are then reflected via the projected image and data. FIG. 10 depicts an exemplary projected image on an anatomical model.

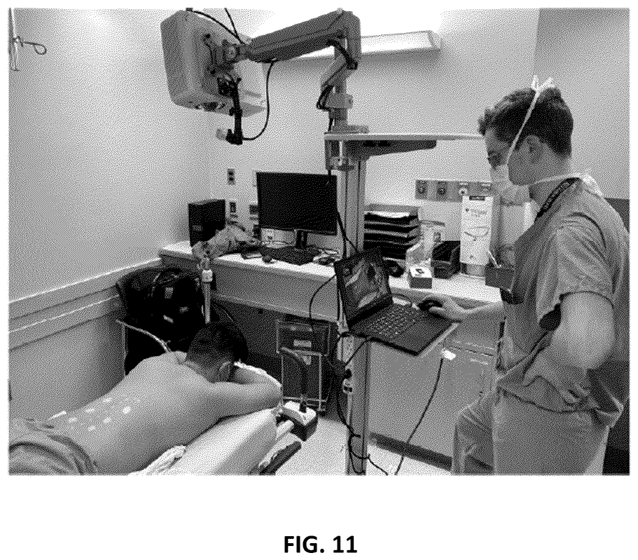

The disclosed system and methods of use may be used in spinal surgery. FIG. 11 depicts the disclosed system in use projecting surgical data onto the body of a subject, e.g., the subject's spine (extracted using the extrusion system and aligned via markerless registration). In some embodiments, the disclosed system projects patient medical data, e.g., slipped discs or misalignments, as well as guidance for device placement, directly onto the body of the patient.

The disclosed system and methods of use may also be used in neurological surgery indications. In some embodiments, images of tumor placement in a patient may be added to the projection for easier navigation. In some embodiments, the disclosed system is able to be used for guidance in deep brain stimulator placement.

While various embodiments have been described herein, it should be understood that they have been presented by way of example only, and not limitation. For example, there are several other indications for use, such as maxillofacial procedures, pulmonology, urology, etc., wherein the disclosed system can be used to aid in surgical and non-surgical procedures. Numerous other indications for use exist, and are considered to be included in this disclosure. Furthermore, although various embodiments have been described as having particular features and/or combinations of components, other embodiments are possible having a combination of any features and/or components.

Where methods and steps described above indicate certain events occurring in a certain order, the ordering of certain steps may be modified. Additionally, certain of the events may be performed repeatedly concurrently in a parallel process when possible, as well as performed sequentially as described above. Furthermore, certain embodiments may omit one or more described events.

Where methods are described, it should be understood that such methods can be computer-implemented methods. Similarly stated, a non-transitory processor readable medium can store code representing instructions configured to cause a processor to cause the described method to occur or be carried out. For example, an instrument can include a processor and a memory and can cause one or more method steps described herein to occur. Thus, some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes.

Although various embodiments have been described as having particular features and/or combinations of components, other embodiments are possible having any combination or sub-combination of any features and/or components from any of the embodiments described herein.

1. A non-transitory, processor-readable medium storing code, the code including instructions to cause the processor to:
receive, from an optical sensor, data associated with an operative field that includes a subject;
access a three-dimensional (3D) virtual model associated with at least one of the subject or an object in the operative field;
define an observed mesh that includes a representation of the subject, based on the data received from the optical sensor;
define a virtual 3D environment, including the virtual model;
register, in a virtual 3D environment, the virtual model to the observed mesh or the observed mesh to virtual model; and
project, in real time, a rendering of the virtual model into the operative field such that the rendering of the virtual model is scaled and oriented relative to the at least one of the subject or the object in the real-world operative field as it appears in the virtual 3D environment.

2. (canceled)

3. The non-transitory, processor-readable medium of claim 1, the code further comprising instructions to cause the processor to:
define a virtual camera in the virtual 3D environment, the virtual camera having virtual intrinsic parameters that match physical intrinsic parameters of a physical projector;
register the physical projector to the virtual camera based on a known spatial relationship between the optical sensor and the physical projector such that extrinsic parameters of physical projector, including location and orientation relative to the subject, match extrinsic parameters of the virtual camera, such as location and orientation relative to the observed mesh; and
create the rendering of the virtual model based on a field of view of the virtual camera.

4. (canceled)

5. The non-transitory, processor-readable medium of claim 1, wherein the 3D model is based on intraoperative medical imaging of the subject.

6. The non-transitory, processor-readable medium of claim 1, wherein:
the 3D model is based on pre-operative medical imaging of the subject; and
projecting the rendering of the virtual model includes projecting internal anatomical information associated with the pre-operative medical imaging onto a skin surface of the subject.

7. The non-transitory, processor-readable medium of claim 1, the code further comprising instructions to cause the processor to:
annotate the virtual 3D environment with at least one of preoperative trajectory plans, preoperative user annotations, or intraoperative user annotations; and
project, in real time and with the rendering of the virtual model, the at least one of preoperative trajectory plans, preoperative user annotations, or intraoperative user annotations.

8. (canceled)

9. (canceled)

10. The non-transitory, processor-readable medium of claim 1, wherein:
data associated with the operative field is continuously received from the optical sensor; and
the rendering of the virtual model is continuously updated in real time such that a position, an orientation, and a scale of the rendering of the virtual model is updated based on changes of at least one of the subject or the object in the operative field.

11. The non-transitory, processor-readable medium of claim 1, wherein:
the data received from the optical sensor includes an indication of a surgical tool; the virtual model is associated with the surgical tool, the code further comprising instructions to cause the processor to:
continuously update the virtual 3D environment to track a position and an orientation of a virtual surgical instrument corresponding to the surgical tool based on data received from the optical sensor.

12. The non-transitory, processor-readable medium of claim 1, wherein:
the data received from the optical sensor includes an indication of a surgical tool; the virtual model is associated with the surgical tool, the code further comprising instructions to cause the processor to:
define a virtual camera in the virtual 3D environment, the virtual being associated with the surgical tool and having a position and an orientation associated with a position and an orientation of the surgical tool;
create the rendering of the virtual model based on a field of view of the virtual camera; and
continuously update the virtual 3D environment to track the position and the orientation of a virtual surgical instrument corresponding to the surgical tool based on data received from the optical sensor such that the rendering of the virtual model is updated in real time based on the position and the orientation of the surgical tool.

13. The non-transitory, processor-readable medium of claim 1, wherein the virtual model is a first virtual model and the virtual 3D environment includes a second virtual model associated with the subject, the code further comprising instructions to cause the processor to:
define a virtual camera in the virtual 3D environment; and
create the rendering of the virtual first model based on a field of view of the virtual camera, the second virtual model being transparent to the virtual camera such that a rendering of the subject is not projected onto the subject.

14. (canceled)

15. A method, comprising:
receiving, from an optical sensor, data associated with an operative field that includes a subject and a surgical tool;
accessing a three-dimensional (3D) virtual model associated with the surgical tool;
defining an observed mesh that includes a representation of the subject and a representation of the surgical tool based on the data received from the optical sensor;
registering (i) the observed mesh to a virtual 3D environment that includes the 3D virtual model associated with the surgical tool and a 3D virtual representation of the subject or (ii) the virtual 3D environment to the observed mesh;
defining a virtual camera in the virtual 3D environment, the virtual camera having a position and an orientation associated with a position and an orientation of the 3D virtual model of the surgical tool; and
projecting, in real time, a rendering of a virtual object such that the rendering of the virtual object is scaled and oriented based on the position and the orientation of the surgical tool.

16. The method of claim 15, further comprising:
accessing pre-operative medical imaging of the subject;
defining the virtual object based on the pre-operative medical imaging, the virtual object being a 3D representation of a sub-surface anatomical feature of the subject, such that the rendering of the virtual object is projected onto a surface of the subject from the point of view of the surgical tool.

17. The method of claim 15, wherein:
the surgical tool is tracked in real time by the optical sensor using a plurality of fiducial markers coupled to the surgical tool;
the representation of the surgical tool in the observed mesh is based on the plurality of fiducial markers; and
the representation of the surgical tool in the observed mesh is registered to the virtual 3D environment based on data associated with the plurality of fiducial markers received from the optical sensor.

18. (canceled)

19. The method of claim 15, wherein the virtual camera is a first virtual camera, the method further comprising:
projecting, in the virtual 3D environment and from the point of view of the first virtual camera, a virtual annotation associated with a virtual object onto a surface of the 3D virtual representation of the subject;
defining a second virtual camera, the second virtual camera having virtual intrinsic and extrinsic parameters that match physical intrinsic and extrinsic parameters of a physical projector; and
creating the rendering of the virtual object based on a field of view of the second virtual camera, the virtual object including the virtual annotation.

20. The method of claim 18, wherein the virtual annotation includes a representation of sub-surface anatomy.

21. The method of claim 20, further comprising:
receiving data associated with at least one of a preoperative or intraoperative medical imaging of the subject;
defining an anatomical model of the subject including the representation of the sub-surface anatomy.

22. An apparatus, comprising:
a housing;
an optical sensor disposed within the housing;
a projector disposed within the housing;
a processor operatively coupled to the optical sensor and the projector; the processor configured to:
receive data from the optical sensor that associated with an operative field;
define a virtual three-dimensional (3D) environment including a virtual representation of the subject and an annotation;
register data received from the optical sensor to the virtual 3D environment or the virtual 3D environment to the data received from the optical sensor;
send a signal to the projector to cause the projector to project a rendering of at least a portion of the virtual 3D environment that includes the annotation onto a surface of the subject.

23. (canceled)

24. The apparatus of claim 22, wherein the processor is configured to project the rendering of the virtual 3D environment onto the surface of the subject in real time such that annotation is scaled and oriented relative to the subject in the operative field as it appears in the virtual 3D environment.

25. The apparatus of claim 22, wherein defining the virtual 3D environment includes defining the annotation associated with a model of the subject's sub-surface anatomy based on the medical imaging;

26. The apparatus of claim 22, wherein:
the processor and the optical sensor are collectively configured to identify a position and an orientation of a surgical tool in the operative field based on fiducial markers coupled to the surgical tool;
receiving data associated with at least one of preoperative or intraoperative medical imaging of the subject;
defining the virtual 3D environment includes defining the annotation associated with a model of the subject's sub-surface anatomy based on the medical imaging;
registering includes registering the position and the orientation of the surgical tool to a virtual representation of the surgical tool within the virtual 3D environment, the processor further configured to:
virtually project, within the virtual 3D environment and from the point of view of the virtual representation of the surgical tool, the annotation onto a virtual surface of a virtual representation of the subject;
define a virtual camera having virtual intrinsic and extrinsic parameters that match physical intrinsic and extrinsic parameters of the projector,
render the at least the portion of the virtual 3D environment from the point of view of the virtual camera.

27. The apparatus of claim 26, wherein the annotation is virtually orthographically projected onto the virtual surface of the virtual representation of the subject.
